(19) United States
(12) Patent Application Publication
(10) Pub. No.: US 2007/0050213 A1
(43) Pub. Date: Mar. 1, 2007

(54) Authentication processing method and apparatus

(75) Inventors: Tetsuya Matsushima, Kawasaki (JP); Seigo Kotani, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 11/295,657

(22) Filed: Dec. 7, 2005

(30) Foreign Application Priority Data
Sep. 1, 2005 (JP) 2005-253304

(51) Int. Cl. G06F 19/00; A61B 5/00

(52) U.S. Cl. 705/3

(57) ABSTRACT

This invention is to provide a technique to appropriately authenticate the requesting side and the diagnosis side in the remote diagnosis system. In this invention, an authentication server communicating with a diagnosis requesting side terminal and a diagnosis side terminal carries out: at the beginning of a remote diagnosis, judging whether or not an image obtained in the diagnosis requesting side terminal satisfies a first condition; at the beginning of the remote diagnosis, judging whether or not an image obtained by the diagnosis side terminal satisfies a second condition; and upon detection that affirmative judgments are obtained in the first and second judgings, generating authentication data including information concerning the diagnosis requesting side terminal, information concerning the diagnosis side terminal and a diagnosis time. Accordingly, it is possible to guarantee that both terminals have an appropriate capability and etc.

### TECHNICAL FIELD OF THE INVENTION

This invention relates to an authentication processing technique in a remote diagnosis system.

### BACKGROUND OF THE INVENTION

A system tries to be introduced in which a clinic, hospital or patient house in a remote location is connected with a specialized hospital or the like to carry out a bidirectional real-time image communication therebetween, and a specialist carries out diagnosis based on received image information relating to the patient in the remote location.

For example, US 2005/0021375 discloses a system in which a hospital site on a requesting side, a hospital side on a diagnosis side and a center site, which judges image capability, are provided. Specifically, according to request content on the requesting side, the center site sends necessary test image to the diangosis side, and receives its display result information to judge the image display capability on the diagnosis side.

In the aforementioned publication, only the image display capability on the diagnosis side is guaranteed, and the image taken on the requesting side is not guaranteed. In a case of a medical image whose image capability is determined by a physical condition within a photographing apparatus such as an X-ray CT or MRI, such a technique is sufficient. However, in a case where an image such a bidirectionally communicated image, endoscope image or the like, which is used in a case of the interactive remote diagnosis with the patient, is directly generated under the visible ray environment, and a diagnostician carries out visual diagnosis based on the generated image, unless the photographing environment and capability on the requesting side is guaranteed, the diagnosis itself cannot be guaranteed.

### SUMMARY OF THE INVENTION

Therefore, an object of this invention is to provide a technique to appropriately authenticate the requesting side and the diagnosis side in the remote diagnosis system.

An authentication processing method according to this invention is an authentication processing method executed by an authentication server communicating with a diagnosis requesting side terminal and a diagnosis side terminal, and including: at the beginning of a remote diagnosis carried out between the diagnosis requesting side terminal and the diagnosis side terminal, judging whether or not an image obtained in the diagnosis requesting side terminal satisfies a first condition; at the beginning of the remote diagnosis, judging whether or not an image obtained by the diagnosis side terminal satisfies a second condition; and upon detection that affirmative judgments are obtained in the first and second judgings, generating authentication data including information concerning the diagnosis requesting side terminal, information concerning the diagnosis side terminal and a diagnosis time, and storing the generated authentication information into an authentication data storage. By carrying out the first judging, it is possible to confirm whether or not a photographing environment in which the diagnosis requesting side terminal is installed and a capability of a photographing device are appropriate. In addition, by carrying out the second judging, it is possible to confirm whether or not the display quality in the diagnosis side terminal is appropriate. When the affirmative judgments are obtained in the first and second judgings, it is possible to guarantee that both of the diagnosis requesting side terminal and the diagnosis side terminal have an appropriate capability and are in an appropriate environment. Incidentally, the diagnosis in this invention is not limited to a diagnosis in the medical treatment, and can be applied to diagnoses of a product, a building or the like.

In addition, the authentication processing method according to this invention may further include: carrying out a user authentication processing for a user of the diagnosis requesting side terminal; carrying out a user authentication processing for a user of the diagnosis side terminal; carrying out a device authentication processing for the diagnosis requesting side terminal; and carrying out a device authentication processing for the diagnosis side terminal. In addition, when processing results of the user authentication processing for the user of the diagnosis requesting side terminal and the device authentication processing for the diagnosis requesting side terminal are affirmative, the first judging may be carried out, and when processing results of the user authentication processing for the user of the diagnosis side terminal and the device authentication processing for the diagnosis side terminal are affirmative, the second judging may be carried out. Furthermore, the information concerning the diagnosis requesting side terminal may include an ID of the user of the diagnosis requesting side terminal and an ID of the diagnosis requesting side terminal, and the information concerning the diagnosis side terminal may include an ID of the user of the diagnosis side terminal and an ID of the diagnosis side terminal. Thus, by carrying out the authentication for the user and the terminal, not only the confirmation that the capability and the environment of the diagnosis requesting side terminal and the diagnosis side terminal are appropriate, but also evidence data concerning what device is used by what person (for example, a patient and medical care personnel) are recorded.

Furthermore, the carrying out the user authentication processing for the user of the diagnosis requesting side terminal or for the user of the diagnosis side terminal may include transmitting an authentication request to a server that carries out the user authentication processing; and receiving a processing result of the user authentication processing from the server that carries out the user authentication processing. In addition, the carrying out the device authentication processing for the diagnosis requesting side terminal or for the diagnosis side terminal may include transmitting an authentication request to a server that carries out a device authentication processing; and receiving a processing result of the device authentication from the server that carries out the device authentication processing. This is because a form in which the authentication authority site is separately provided is dealt with.

Furthermore, the aforementioned first judging may include: searching a device data storage storing information concerning a terminal whose obtained image was judged to be satisfied with the first or second condition, and information concerning a valid period of the judgment, to confirm whether or not the judgment as to the diagnosis requesting side terminal is carried out within the valid period. In addition, the aforementioned second judging may include: searching the device data storage to confirm whether or the judgment as to the diagnosis side terminal is carried out within the valid period. When the judgment is carried out within the valid period, the load to actually confirm the image data can be reduced.

In addition, the aforementioned first judging may include: receiving image data photographed by a camera connected to the diagnosis requesting side terminal from the diagnosis requesting side terminal; calculating a value of a predetermined image feature amount; and confirming whether or not the value of the predetermined image feature amount is within a prescribed range. Moreover, the aforementioned first judging may include: receiving a value of a predetermined image feature amount calculated from image data photographed by a camera connected to the diagnosis requesting side terminal; and confirming whether or not the value of the predetermined image feature amount is within a prescribed range.

Furthermore, the aforementioned second judging may include: receiving image quality information obtained by an image sensor connected to the diagnosis side terminal from the diagnosis side terminal; calculating a value of a predetermined image feature amount by using the image quality information; and confirming whether or not the value of the predetermined image feature amount is within a prescribed range. In addition, the aforementioned second judging may include: receiving a value of a predetermined image feature amount calculated from image quality information obtained by an image sensor connected to the diagnosis side terminal from the diagnosis side terminal; and confirming whether or not the value of the predetermined image feature amount is within a prescribed range.

Incidentally, the predetermined image feature amount may be a color temperature.

In addition, the authentication processing method according to this invention may further include: carrying out an environment authentication processing for the diagnosis requesting side terminal; and carrying out an environment authentication processing for the diagnosis side terminal. The environment authentication processing is an authentication to check whether appropriate programs are installed, whether a predetermined dangerous program is not installed or the like. Moreover, when a processing result of the environment authentication processing for the diagnosis requesting side terminal is affirmative, the first judging may be carried out, and when a processing result of the environment authentication processing for the diagnosis side terminal is affirmative, the second judging may be carried out.

It is possible to create a program for causing a computer to execute the authentication processing method according to this invention, and this program is stored in a storage medium or a storage device such as a flexible disk, a CD-ROM, an optical magnetic disk, a semiconductor memory, and a hard disk. Further, the program may be distributed as a digital signal through a network. Incidentally, intermediate processing results are temporarily stored in a storage device such as a main memory.

### BRIEF DESCRIPTION OF THE DRAWINGS

FIG. 1 is a diagram showing a system outline in an embodiment of this invention;

FIG. 2 is a diagram showing a first portion of a processing flow according to the embodiment of this invention;

FIG. 3 is a diagram showing an example of data stored in a device DB;

FIG. 4 is a diagram to explain photographing of the test sample;

FIG. 5 is a diagram showing an example of a color temperature map;

FIG. 6 is a diagram showing a second portion of the processing flow according to the embodiment of this invention;

FIGS. 7A and 7B are diagrams to explain obtainment of image quality information;

FIG. 8 is a diagram showing a third portion of the processing flow according to the embodiment of this invention;

FIG. 9 is a diagram showing an example of data stored in a color authentication DB;

FIG. 10 is a functional block diagram of a computer.

### DETAILED DESCRIPTION OF THE PREFERRED EMBODIMENTS

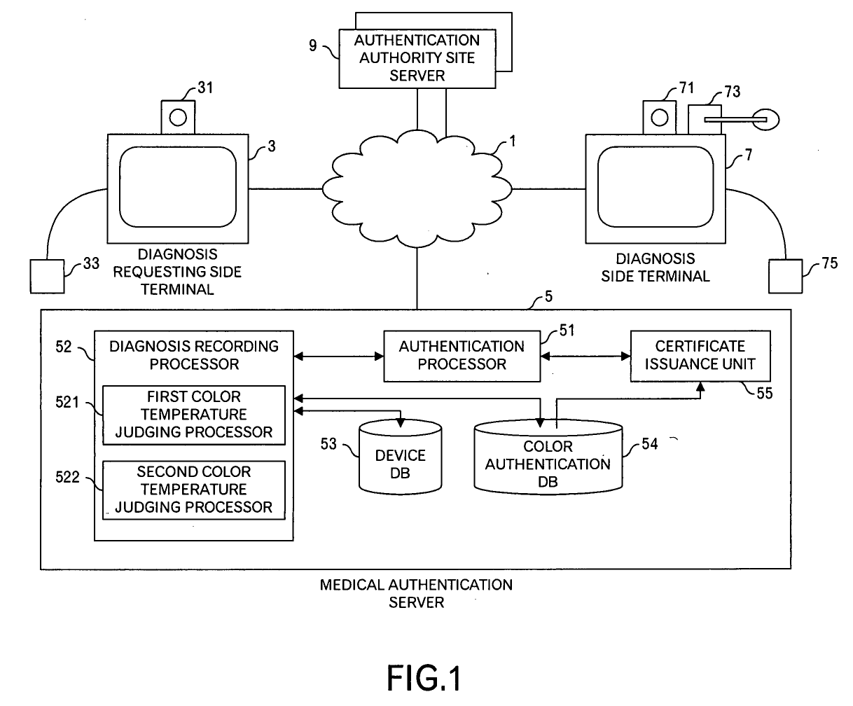

FIG. 1 is a system outline diagram according to an embodiment of this invention. In FIG. 1, a network 1 is connected with one or plural diagnosis requesting side terminals 3, a medical authentication server 5, one or plural diagnosis side terminals 7 and one or plural authentication authority site servers 9. In the following, a telemedicine system is explained as an example. However, this embodiment can also be applied to fields other than the medical field.

The diagnosis requesting side terminal 3 may be provided in a patient house in addition to a clinic or a small-scale hospital, and carries out interactive image communication with the diagnosis side terminal 7. Medical care personnel such as doctors or nurses in the clinic or the like operate the diagnosis requesting side terminal 3, and the patient may individually operate this terminal. In addition, the diagnosis requesting side terminal 3 includes a video camera 31, an authentication information obtaining unit 33, a display device to display information such as images, and a communication unit to communicate through the network 1. The authentication information obtaining unit 33 is a device to read out a fingerprint, a vein pattern, an iris or the like of the user, or a semiconductor chip that collects and manages device information (mainly hardware information such as a manufacturer, manufactured date, model number, serial number, certification information, device configuration and the like) of the diagnosis requesting side terminal 3, and environment information (mainly software information such as a type of an operating system (OS), and information indicating whether or not invasion of dangerous programs such as viruses and spyware).

The diagnosis side terminal 7 is provided in a specialized hospital or the like, and is basically operated by a doctor who carries out the diagnosis. This diagnosis side terminal 7 has a vide camera 71, a display device to display information such as images, a image sensor 73 to obtain image quality information to confirm the quality of images displayed on the display device, and an authentication information obtaining unit 75 having the same functions as the authentication information obtaining unit 33.

The authentication authority site server 9 includes a server that carries out a user authentication processing such as an authentication using an ID and a password, and a biometrics authentication using the fingerprint, the vein pattern, the iris or the like, a server that carries out a device authentication processing, a server that carries out an environment authentication processing and the like.

The medical authentication server 5 has an authentication processor 51 that carries out a user authentication processing, a device authentication processing and/or an environment authentication processing by itself, or carries out various kinds of authentication processings in cooperation with the authentication authority site server 9, a diagnosis recording processor 52 having a first color temperature judging processor 521 and a second color temperature judging processor 522 and generating color authentication data, a device DB 53 storing information concerning a terminal whose color temperature has been judged, a color authentication DB 54 storing the color authentication data generated by the diagnosis recording processor 52, and a certificate issuance unit 55 that issues the color authentication data stored in the color authentication DB 54 as certificate data in response to a request.

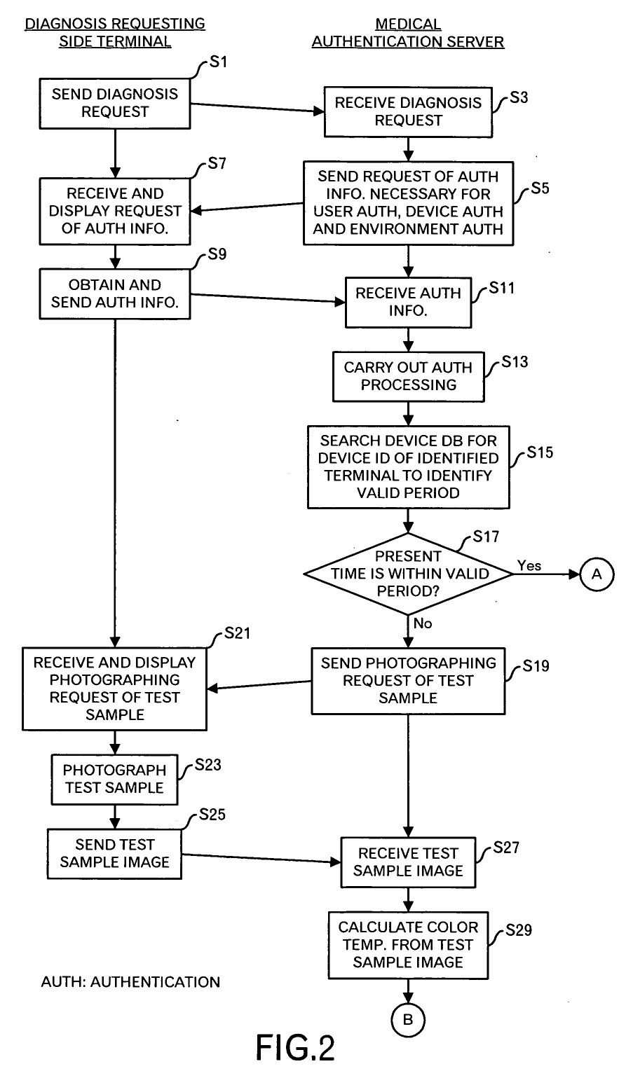

Next, processing content of the system shown in FIG. 1 will be explained with reference to FIGS. 2 to 9. First, the user (the patient or medical care personnel in the clinic or the like) of the diagnosis requesting side terminal 3 operates the diagnosis requesting side terminal 3 to cause it to transmit a diagnosis request including, for example, an ID of the diagnosis request destination and the like to the medical authentication server 5 (step S1). The authentication processor 51 of the medical authentication server 5 receives the diagnosis request including the ID of the diagnosis request destination and the like from the diagnosis requesting side terminal 3 (step S3), and replies a request of authentication information necessary for an authentication processing(s) to be carried out among the user authentication processing, the device authentication processing and the environment authentication processing, to the diagnosis requesting side terminal 3 (step S5). In this embodiment, at least the user authentication and the device authentication are carried out.

The diagnosis requesting side terminal 3 receives the request of the authentication information from the medical authentication server 5 and displays the received request on the display device (step S7). For example, the terminal 3 prompts the patient to input the ID and the password of the patient. In addition, in a case where the diagnosis requesting side terminal 3 is provided in the clinic or the like, the terminal 3 may also prompt the medical care personnel to input the ID and the password of the medical care personnel in the clinic or the like. Moreover, in a case where the biometric authentication processing is carried out, the terminal 3 requests the authentication information obtaining unit 33 to read out the fingerprint, the vein pattern or the like. Furthermore, in a case where the device authentication processing or the environment authentication processing is carried out, the terminal 3 displays information representing that the output of data necessary for such an authentication processing is requested to the authentication information obtaining unit 33, for the user.

The diagnosis requesting side terminal 3 accepts inputs of the ID(s) and the password(s) of the patient, or the patient and the medical care personnel. When the biometric authentication processing is carried out, the authentication information obtaining unit 33 of the diagnosis requesting side terminal 3 reads out the fingerprint data, the vein pattern or the like. Incidentally, if necessary, predetermined feature data may be extracted from the read data. Furthermore, the authentication information obtaining unit 33 collects the authentication information necessary for the device authentication processing and the environment authentication processing when they are carried out. Then, the diagnosis requesting side terminal 3 transmits the obtained authentication information to the medical authentication server 5 (step S9).

The authentication processor 55 of the medical authentication server 5 receives the authentication information from the diagnosis requesting side terminal 3 (step S11), and carries out predetermined authentication processings corresponding to the received authentication information when it carries out the authentication processings to obtain authentication results. In addition, when the medical authentication server 5 requests actual authentication processings to the authentication authority site server 9, the medical authentication server 5 transmits an authentication request including the authentication information to the authentication authority site server 9. Incidentally, when the authentication information which can be dealt with by each of the authentication authority site servers 9 is different, the authentication request including the pertinent authentication information may be transmitted to the corresponding authentication authority site server 9 among the plural authentication authority site servers 9. Then, the authentication processor 51 of the medical authentication server 5 receives the authentication processing results from the authentication authority site servers 9, and totally judges whether or not the authentication processings are successfully completed (step S13). When any one of the authentication processing results indicates the failure, the authentication processor 51 judges that the entire authentication processing is also failed, and notifies the diagnosis requesting side terminal 3 of the authentication failure. The details of the individual authentication processing are not main portions of this embodiment. Therefore, the further explanation is omitted. In the following, a case where the authentication processing is successfully completed will be explained. In addition, the authentication processor 51 outputs an authentication success notice including a device ID of the diagnosis requesting side terminal 3, which is included in the authentication information for the device authentication processing, the ID of the patient or the IDs of the patient and the medical care personnel, which are included in the authentication information for the user authentication processing, to the diagnosis recording processor 52.

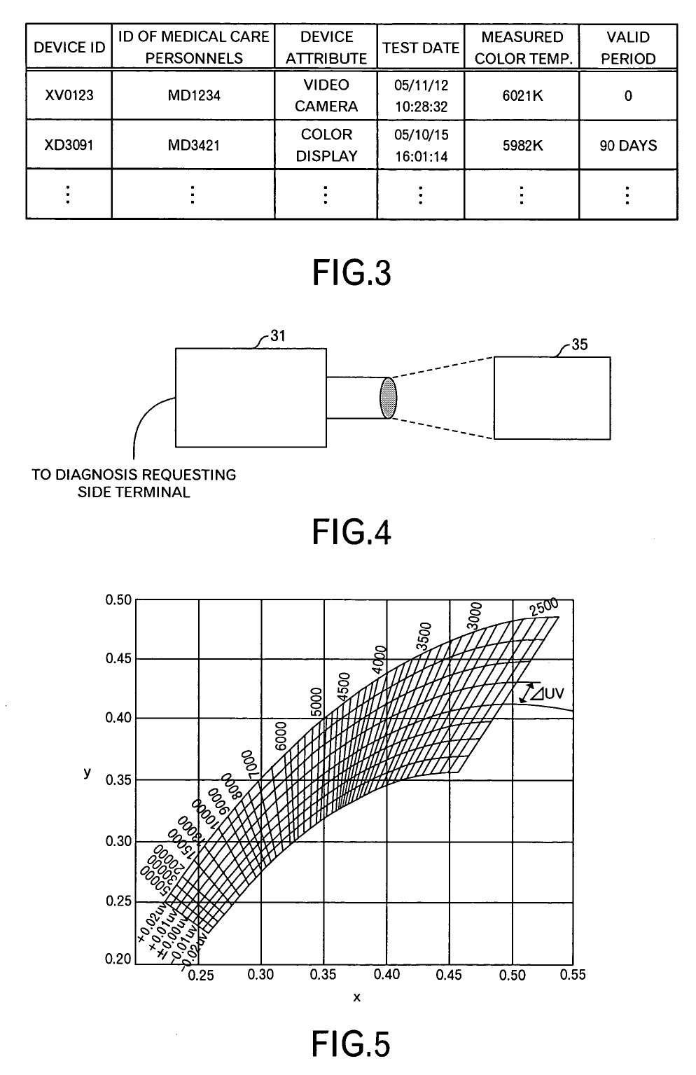

Next, the diagnosis recording processor 52 searches the device DB 53 by the identified device ID of the diagnosis requesting side terminal 3, and identifies a valid period of a color temperature measurement test described below (step S15). The device DB 53 stores data as shown in FIG. 3. A table of FIG. 3 stores, for each device ID, an ID (an ID of the medical care personnel, or an ID of the patient) of the user involving the color temperature measurement test by operating the terminal of the device ID, a device attribute indicating whether or not the vide camera of the diagnosis requesting side terminal 3 was a target of the color temperature measurement test or the display device (color display) of the diagnosis side terminal 7 was a target of the color temperature measurement test, a test date of the color temperature measurement test, a measured color temperature, a valid period (or expiration date). Incidentally, when two or more records relating to the same device are detected in the step S15, the latest record is employed in a case of the same device attribute, and in a case where the device attribute is different, a record whose device attribute is the video camera is employed as for the diagnosis requesting side terminal 3 as the transmission source of the diagnosis request. In addition, when the medical authentication server 5 is firstly used, the device ID of the diagnosis requesting side terminal 3 is not registered in this table.

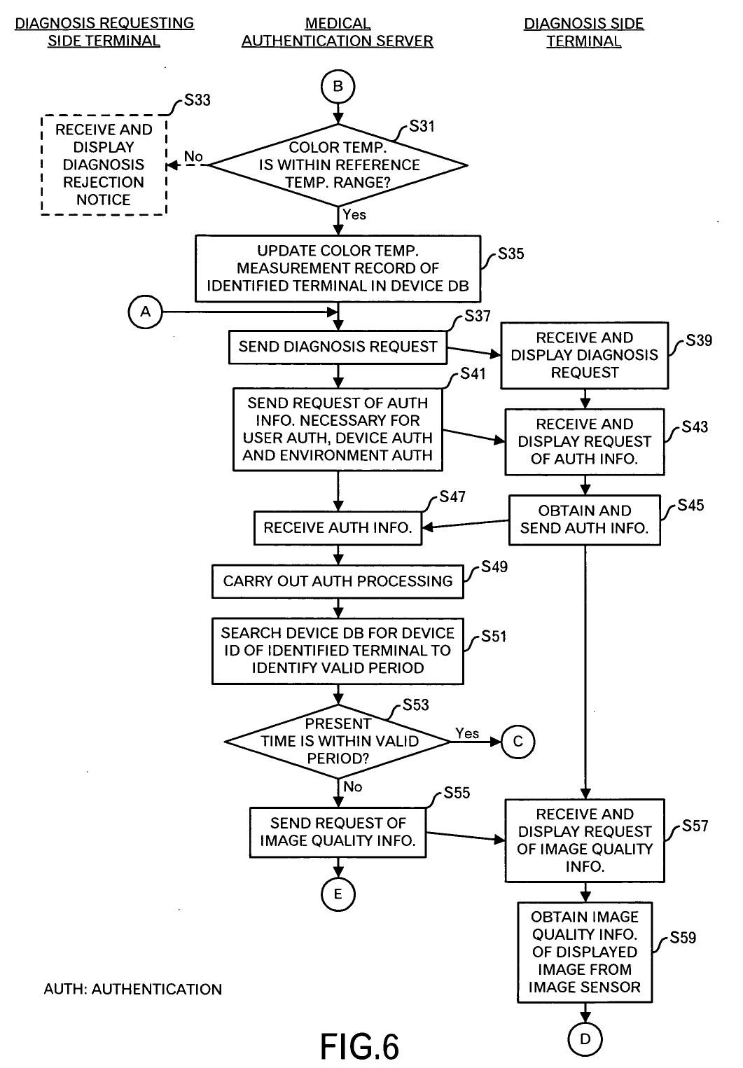

Then, the diagnosis recording processor 52 confirms whether or not the present time is within the valid period identified in the step S15 (step S17). When the present time is within the valid period, the processing shifts to step S37 of FIG. 6 through a terminal A. On the other hand, when the present time is not within the valid period, or the expiration date is not registered, the diagnosis recording processor 52 transmits a photographing request of a test sample to the diagnosis requesting side terminal 3 (step S19).

The diagnosis requesting side terminal 3 receives the photographing request of the test sample from the medical authentication server 5, and displays the request on the display device (step S21). As shown in FIG. 4, the user (the patient or the medical care personnel) of the diagnosis requesting side terminal 3 operates the video camera 31 connected to the diagnosis requesting side terminal 3 to cause the vide camera 31 to photograph a predetermined test sample 35. The test sample 35 is basically a white plane. However, because the normally sold sample is sufficient, the further explanation is omitted. The video camera 31 photographs the test sample 35 in response to an instruction of the user (step S23), and transmits a photographed test sample image to the medical authentication server 5 (step S25). The test sample image is a static image. The diagnosis recording processor 52 of the medical authentication server 5 receives the test sample image from the diagnosis requesting side terminal 3 (step S27), and the first color temperature judging unit 521 calculates the color temperature from the received test sample image, and stores the calculated color temperature into a storage device such as a main memory (step S29).

The calculation of the color temperature is carried out as follows. For each pixel value (R, G, B) of the test sample image, x and y are calculated by the following equations.
x=0.6×R−0.28×G−0.32×B
y=0.2×R−0.52×G+0.31×B
These x and y represent a point on a color temperature map shown in FIG. 5. An ideal object that is a black body (that is, a perfect radiator) completely absorbing the energy changes the color of its light when the temperature heightens, and the absolute temperature T [K] at this time is called as the color temperature. FIG. 5 shows the color temperature and a path (black body path) of the color, isothermal color temperature lines, and isanormals, which are apart from the black body path by the same deviation (Δuv). Specifically, +−0.01 represents the black body path, and isanormals whose deviations are +0.01uv, +0.02uv, −0.01uv and −0.02uv, and isothermal color temperature lines (2300K to 50000K) connecting points of the same color temperature with respect to the black body path and isanormals are drawn. Therefore, for example, when x=0.45 and y=0.37, the color temperature is detected as about 2500K on the isanormal of the deviation −0.01uv. Incidentally, when the values of x and y are calculated, the corresponding color temperature is calculated from the black body path, the isothermal color temperature line and the isanormals by carrying out the interpolation if necessary. Such a calculation of the color temperature is well known. Therefore, the further explanation is omitted. Incidentally, because the color temperature is calculated for each pixel, a statistical value such as an average value is calculated, and the statistical value is used as the color temperature of the diagnosis requesting side terminal 7. In addition, an average value of x and an average value of y may be calculated, and the color temperature may be calculated from the average value of x and the average value of y. In this case, the average value may be changed to another statistical value.

The processing shifts to a processing in FIG. 6 through a terminal B. Then, the first color temperature judging processor 521 judges whether or not the color temperature is within a prescribed reference temperature range (step S31). When it is judged that the color temperature is not within the prescribed reference temperature range, the first color temperature judging processor 521 transmits a diagnosis rejection notice representing the color temperature is out of the reference temperature range to the diagnosis requesting side terminal 3. The diagnosis requesting side terminal 3 receives the diagnosis rejection notice from the medical authentication server and displays the notice on the display device (step S33).

On the other hand, when it is judged that the color temperature is within the prescribed reference temperature range, the first color temperature judging processor 521 updates the color temperature measurement record of the terminal identified by the device ID in the device DB 53 (step S35). Specifically, a record including a device ID, a user ID (the patient ID, or the medical care personnel ID) of the diagnosis requesting side terminal 3, a device attribute (video camera because of the diagnosis requesting side terminal that has transmitted the diagnosis request), a test date (photographing date of the test sample image, or judgment date), a color temperature calculated in the step S29, a valid period (for example, 0 day) is generated to register it in the device DB 53. In a case of the diagnosis requesting side terminal 3 that has transmitted the diagnosis request, it may be provided in the patient house. Therefore, it is predicted that the image quality is changed every time. Therefore, 0 day is set to the valid period, basically. Incidentally, when it can be confirmed that the diagnosis requesting side terminal 3 is located in the reliable clinic or the like from the device ID, other number of days may be set. In addition, when the type of the installation location or the like is identified from a code structure of the device ID, the type of the installation location of the diagnosis requesting side terminal 3 is identified, and by referring to a table in which the type of the installation location and the valid period are associated, the valid period corresponding to the type of the identified installation location may be identified.

After that, the diagnosis recording processor 52 outputs a notice representing a processing for the diagnosis requesting side terminal 3 to the authentication processor 51. Then, the authentication processor 51 identifies an address of the network 1 from the ID and the like of the diagnosis request destination included in the diagnosis request from the diagnosis requesting side terminal 3, and transmits the diagnosis request to the diagnosis side terminal 7 identified as the diagnosis request destination (step S37). Incidentally, not based on the diagnosis request from the diagnosis requesting side terminal 3, an appropriate diagnosis side terminal 7 may be automatically selected. The diagnosis terminal 7 receives the diagnosis request and displays it on the display device (step S39).

Furthermore, the authentication processor 51 transmits a request of the authentication information necessary for an authentication processing to be carried out among the user authentication processing, the device authentication processing, and the environment authentication processing. In this embodiment, at least the user authentication and the device authentication are carried out.

The diagnosis side terminal 7 receives the request of the authentication information from the medical authentication server 5, and displays the request on the display device (step S43). For example, when the diagnosis side terminal 7 is provided in the specialized hospital or the like, the diagnosis side terminal 7 requests the medical care personnel in the specialized hospital or the like to input the ID and the password. In addition, when the biometric authentication processing is carried out, it requests the user to cause the authentication information obtaining unit 75 to read out the fingerprint, the vein pattern or the like. Furthermore, when the device authentication processing or the environment authentication processing is carried out, it displays information representing the request for the authentication information obtaining unit 75 to output data necessary for such an authentication processing.

The diagnosis side terminal 7 accepts inputs of the ID and password of the medical care personnel. When the biometric authentication processing is carried out, the authentication information obtaining unit 75 of the diagnosis side terminal 7 reads out the fingerprint data, the vein pattern or the like. Incidentally, predetermined feature data may also be extracted from the read data if necessary. Furthermore, the authentication information obtaining unit 75 collects the authentication information necessary for the device authentication processing or the environment authentication processing when they are carried out. Then, the diagnosis side terminal 7 transmits the obtained authentication information to the medical authentication server 5 (step S45).

The authentication processor 51 of the medical authentication server 5 receives the authentication information from the diagnosis side server 7 (step S47), and when the authentication processing is carried out by itself, it carries out a predetermined authentication processing corresponding to the received authentication information to obtain the authentication result. In addition, when the actual authentication processing is requested to the authentication authority site server 9, an authentication request including the authentication information is transmitted to the authentication authority site server 9. Incidentally, when the authentication information, which can be handled by the authentication authority site server 9, is different among the plural authentication authority site servers 9, the authentication request including the specific authentication information may be transmitted to the corresponding authentication authority site server 9. Then, the authentication processor 51 of the medical authentication server 5 receives the processing results of the authentication processings from the authentication authority site server 9, and judges whether or not the entire authentication processing is successfully completed (step S49). When any one of the authentication results indicates failure, the authentication processor 51 judges that the authentication processing is failed as a whole to notify the diagnosis side server 7 of the authentication failure. The details of the individual authentication processings are not main portions of this embodiment. Therefore, the further explanation is omitted. In the following, a case where the authentication processing is successfully completed is explained. In addition, the authentication processor 51 outputs an authentication success notice including the device ID of the diagnosis side terminal 7, which is included in the authentication information used in the device authentication processing, for example, and the ID of the medical care personnel as the user of the diagnosis side terminal 7, which is included in the authentication information used in the user authentication processing to the diagnosis recording processor 52.

Next, the diagnosis recording processor 52 searches the device DB 53 by the identified device ID of the diagnosis side terminal 7 to identify the valid period of the color temperature measurement test (step S51). Incidentally, when the two or more records relating to the same device ID are extracted in the step S51, the latest record is employed in a case of the same device attribute, and in a case where the device attribute is different, the record is employed in which the device attribute for the diagnosis side terminal 7 as the transmission destination indicates the display. In addition, when the medical authentication server 5 is firstly used, the device ID of the diagnosis side terminal 7 may not be registered.

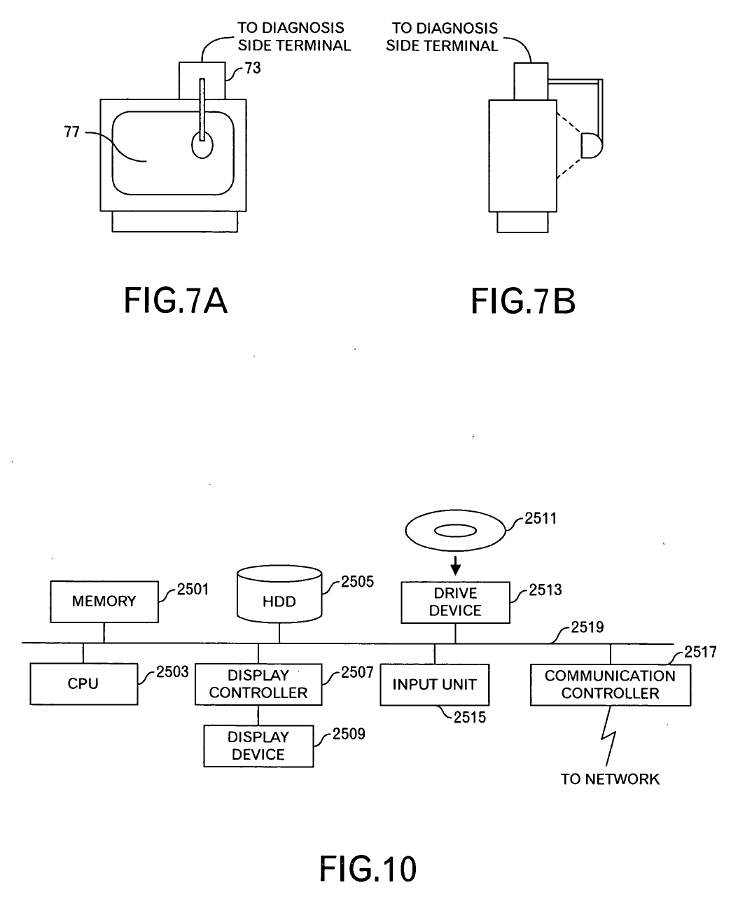

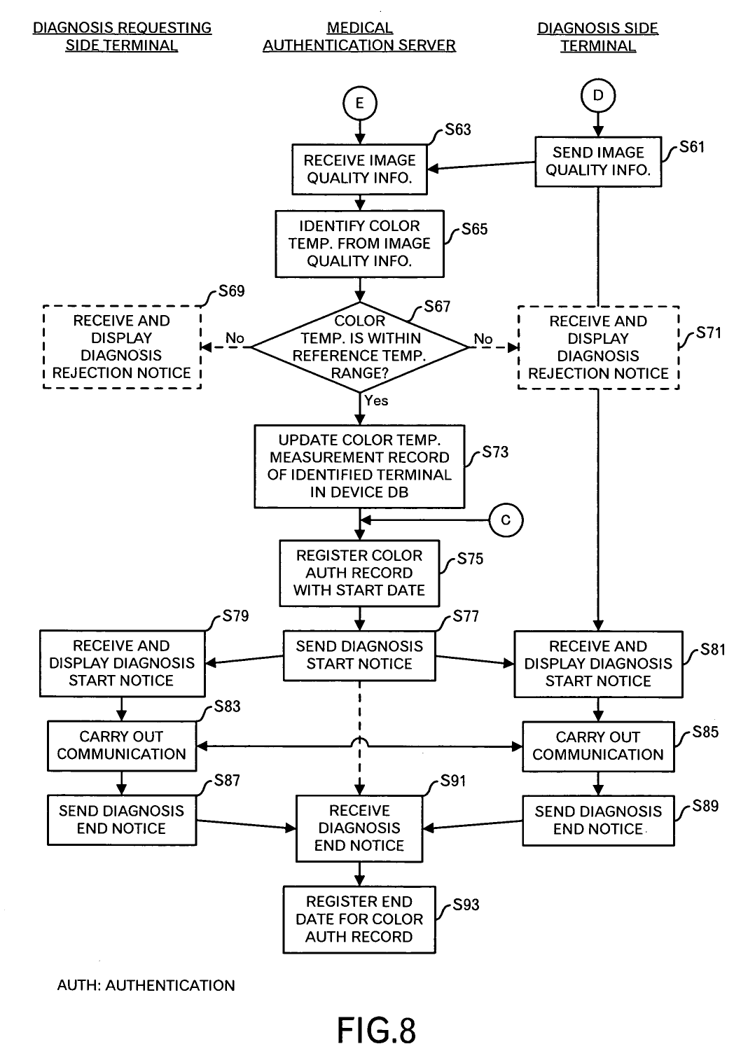

Then, the diagnosis recording processor 52 confirms whether or not the present time is within the valid period identified in the step S51 (step S53). When the present time is within the valid period, the processing shifts to the step S75 in FIG. 8 through the terminal C. On the other hand, when the present time is not within the valid period, or the valid period is not registered, the diagnosis recording processor 52 transmits an image quality information request to the diagnosis side terminal 7 (step S55). The diagnosis side terminal 7 receives the image quality request from the medical authentication server 5, and displays the request on the display device (step S57). In response to an instruction from the user, for example, the diagnosis side terminal 7 displays an image for the color temperature measurement test on the display device of the diagnosis side terminal 7, and causes the image sensor 73 connected to the diagnosis side terminal 7 to obtain the image quality information (step S59). As shown in a front view of FIG. 7A, and a side view of FIG. 7B, the end portion of the image sensor 73 is moved in front of the display screen 77, and is caused to photograph the image for the color temperature measurement test. The image quality information may be image data photographed, and may be the color temperature calculated in the image sensor 73 or the image sensor 73 and the diagnosis side terminal 7. In addition, x and y in the color temperature map may be calculated. Incidentally, the display device with the image sensor 73 is used for the medical treatment. Therefore, the further explanation is omitted. The processing shifts to a processing in FIG. 8 through terminals E and D.

Shifting to the explanation of the processing of FIG. 8, the diagnosis side terminal 7 transmits the obtained image quality information to the medical authentication server 5 (step S61). Then, the diagnosis recording processor 52 of the medical authentication server 5 receives the image quality information from the diagnosis side terminal 7 (step S63), and the second color temperature judging processor 522 identifies a color temperature from the image quality information (step S65). When the image quality information is the image data, the color temperature is calculated as described in the step S29. When the image quality information is the color temperature, any special processing is not carried out in the step S65. In addition, when the image quality information includes x and y on the color temperature map, the color image is calculated from x and y.

Then, the second color temperature judging processor 522 confirms whether or not the color temperature identified in the step S65 is within a prescribed reference temperature range (step S67). Incidentally, the reference temperature range may be identical with the reference temperature range in the step S31 or different from it. When it is judged that the color temperature is out of the reference temperature range, the diagnosis recording processor 52 transmits a diagnosis rejection notice indicating the diagnosis side terminal 7 does not satisfies the condition of the remote diagnosis to the diagnosis side terminal 7 and the diagnosis requesting side terminal 3. The diagnosis requesting side terminal 3 and the diagnosis side terminal 7 receive the diagnosis rejection notice from the medical authentication server 5, and display the notice on the display device (steps S69 and S71).

On the other hand, when the color temperature is within the reference temperature range, the second color temperature judging processor 522 of the diagnosis recording processor 52 updates the color temperature measurement record for the terminal identified by the device ID in the device DB 53 (step S73). Specifically, a record including the device ID of the diagnosis side terminal 7, the user ID (medical care personnel ID) of the diagnosis side terminal 7, the device attribute (display because of the diagnosis side terminal as the transmission destination of the diagnosis request), the test date (a date when the image quality is obtained, or when the judgment is carried out), the color temperature identified in the step S65, and the valid period (for example, 90 days) is generated to register it into the device DB 53. Because it is supposed that the diagnosis side terminal 7 is provided in the specialized hospital, and the display device is not provided in the environment in which image deterioration of the display device rapidly proceeds, the image quality of the display does not change largely. Therefore, 90 days is set basically. Incidentally, as for the display, which has been judged not to be within the reference temperature range once, it is possible to adjust the valid period, for example to set 0 day to the valid period. In addition, when the type of the display is identified from the device ID, the type of the display of the diagnosis side terminal 7 may be identified by the device ID, and by referring to a table in which the type of the display is associated with the valid period, the valid period may be identified.

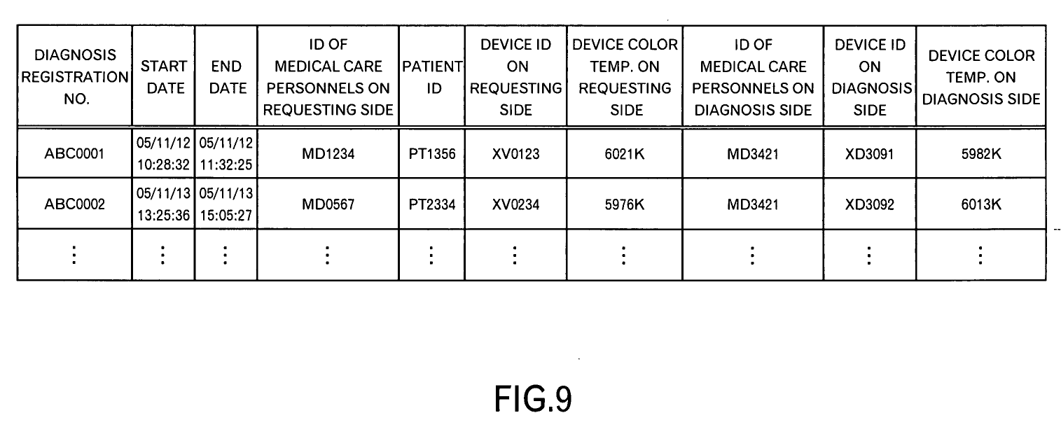

Furthermore, the diagnosis recording processor 52 registers a color authentication record with a start time in the color authentication DB 54 (step S75). An example of data stored in the color authentication DB 54 is shown in FIG. 9. A table of FIG. 9 stores a diagnosis registration number issued for each execution of the step S75, a start date, an end time, an ID of the medical care personnel on the requesting side, an ID of the patient, an ID of the device on the requesting side, a color temperature of the device on the requesting side, an ID of the medical care personnel on the diagnosis side, an ID of the device on the diagnosis side, and the color temperature of the device on the diagnosis side. The start date is a time at the step S75, and the end date is not registered at the step S75. The ID of the medical care personnel on the requesting side is identified from the authentication information for the user authentication processing for the diagnosis requesting side terminal 3, and is registered in a case where the diagnosis requesting side terminal 3 is provided in the clinic or the like. The ID of the patient is identified from the authentication information for the user authentication processing for the diagnosis requesting side terminal 3. The ID of the device on the requesting side is identified from the authentication information for the device authentication processing for the diagnosis requesting side terminal 3. The color temperature of the device on the requesting side is the color temperature calculated at the step S29. The ID of the medical care personnel on the diagnosis side is identified from the authentication information for the user authentication processing for the diagnosis side terminal 7. The ID of the device on the diagnosis side is identified from the authentication information for the device authentication processing for the diagnosis side terminal 7. The color temperature on the diagnosis side is the color temperature identified on the step S65.

After that, the diagnosis recording processor 52 transmits a diagnosis start notice to the diagnosis requesting side terminal 3 and the diagnosis side terminal 7 (step S77). The diagnosis start notice includes addresses of the diagnosis requesting side terminal 3 and the diagnosis side terminal 7, the diagnosis registration number and the like. The diagnosis requesting side terminal 3 and diagnosis side terminal 7 receives the diagnosis start notice from the medical authentication server 5, and displays the notice on the display device (steps S79 and S81). Then, the diagnosis requesting side terminal 3 and the diagnosis side terminal 7 can communicate with each other (steps S83 and S85). Such communication between the terminals is not different from the conventional communication. Therefore, the further explanation is omitted.

After that, the diagnosis requesting side terminal 3 or the diagnosis side terminal 7 transmits a diagnosis end notice including the diagnosis registration number to the medical authentication server 5 (steps S87 or S89). The diagnosis recording processor 52 of the medical authentication server 5 receives the diagnosis end notice from the diagnosis requesting side terminal 3 or the diagnosis side terminal 7 (step S91), and registers the time at the step S91 or the present time as the end date for the color authentication record with respect to the diagnosis requesting side terminal 3 and the diagnosis side terminal 7 in the color authentication DB 54 (step S93). Thus, one record identified by the diagnosis registration number is completed in the color authentication DB 54.

Thus, by the color authentication DB 54, it becomes possible to certificate that the color temperature is verified with respect to both of the diagnosis requesting side terminal 3 and the diagnosis side terminal 7 or that the color temperature is substantially verified. Therefore, for example, even in a case where the validity of the diagnosis in the telemedicine is suspected, it is possible to certificate that there is no problem in the color temperature on the photographing side and the displaying side.

For that, the certificate issuance unit 55 extracts the pertinent record by referring to the color authentication DB 54 in response to a request from the terminal, which is connected to the network 1 and for which the predetermined authentication is carried out, and converts the pertinent record into the certificate data to transmit it to the terminal of the requesting source.

In this way, it becomes possible to wipe out the anxiety on the system in the telemedicine, and to further certificate that the diagnosis is carried out without any problem on the color temperature, later.

Although one embodiment of this invention is described above, this invention is not limited to this. For example, in the aforementioned embodiment, the validity of the terminals is verified based on the color temperature. However, it is possible to judge the validity of the terminal based on another feature data.

In addition, the functional blocks of the medical authentication server 5 shown in FIG. 1 do not always correspond to actual program modules.

In the aforementioned flow chart, as long as the processing result does not change, there are steps, which can be executed in parallel. For example, a set of the authentication processing for the diagnosis requesting side terminal 3, and the color temperature measurement and judging processing may be carried out in parallel with a set of the authentication processing for the diagnosis side terminal 7, and the color temperature measurement and judging processing.

Moreover, it was explained that the authentication information obtaining unit in FIG. 1 has two functions. However, two authentication information obtaining units, each having respective functions, may be provided in both of the diagnosis requesting side terminal 3 and the diagnosis side terminal 7.

In addition, the medical authentication server 5, diagnosis requesting side terminal 3, diagnosis side terminal 7, and the various authentication authority site server 9 are computer devices as shown in FIG. 10. That is, a memory 2501 (storage device), a CPU 2503 (processor), a hard disk drive (HDD) 2505, a display controller 2507 connected to a display device 2509, a drive device 2513 for a removal disk 2511, an input device 2515, and a communication controller 2517 for connection with a network are connected through a bus 2519 as shown in FIG. 28. An operating system (OS) and an application program for carrying out the foregoing processing in the embodiment, are stored in the HDD 2505, and when executed by the CPU 2503, they are read out from the HDD 2505 to the memory 2501. As the need arises, the CPU 2503 controls the display controller 2507, the communication controller 2517, and the drive device 2513, and causes them to perform necessary operations. Besides, intermediate processing data is stored in the memory 2501, and if necessary, it is stored in the HDD 2505. In this embodiment of this invention, the application program to realize the aforementioned functions is stored in the removal disk 2511 and distributed, and then it is installed into the HDD 2505 from the drive device 2513. It may be installed into the HDD 2505 via the network such as the Internet and the communication controller 2517. In the computer as stated above, the hardware such as the CPU 2503 and the memory 2501, the OS and the necessary application program are systematically cooperated with each other, so that various functions as described above in details are realized.

Although the present invention has been described with respect to a specific preferred embodiment thereof, various change and modifications may be suggested to one skilled in the art, and it is intended that the present invention encompass such changes and modifications as fall within the scope of the appended claims.

What is claimed is:

1. An authentication processing method executed by an authentication server communicating with a diagnosis requesting side terminal and a diagnosis side terminal, comprising:
at beginning of a remote diagnosis carried out between said diagnosis requesting side terminal and said diagnosis side terminal, judging whether or not an image obtained in said diagnosis requesting side terminal satisfies a first condition;
at the beginning of said remote diagnosis, judging whether or not an image obtained by said diagnosis side terminal satisfies a second condition; and
upon detection that affirmative judgments are obtained in the first and second judgings, generating authentication data including information concerning said diagnosis requesting side terminal, information concerning said diagnosis side terminal and a diagnosis time, and storing the generated authentication information into an authentication data storage.

2. The authentication processing method as set forth in claim 1, further comprising:
carrying out a user authentication processing for a user of said diagnosis requesting side terminal;
carrying out a user authentication processing for a user of said diagnosis side terminal;
carrying out a device authentication processing for said diagnosis requesting side terminal; and
carrying out a device authentication processing for said diagnosis side terminal, and
wherein said first judging is carried out after it is detected that processing results of said user authentication processing for said user of said diagnosis requesting side terminal and said device authentication processing for said diagnosis requesting side terminal are affirmative,
said second judging is carried out after it is detected that processing results of said user authentication processing for said user of said diagnosis side terminal and said device authentication processing for said diagnosis side terminal are affirmative,
said information concerning said diagnosis requesting side terminal includes an ID of said user of said diagnosis requesting side terminal and an ID of said diagnosis requesting side terminal, and
said information concerning said diagnosis side terminal includes an ID of said user of said diagnosis side terminal and an ID of said diagnosis side terminal.

3. The authentication processing method as set forth in claim 2, wherein said carrying out said user authentication processing for said user of said diagnosis requesting side terminal or for said user of said diagnosis side terminal comprises:
transmitting an authentication request to a server that carries out said user authentication processing; and
receiving a processing result of said user authentication processing from said server that carries out said user authentication processing, and
wherein said carrying out said device authentication processing for said diagnosis requesting side terminal or for said diagnosis side terminal comprises:
transmitting an authentication request to a server that carries out said device authentication processing; and
receiving a processing result of said device authentication processing from said server that carries out said device authentication processing.

4. The authentication processing method as set forth in claim 1, wherein said first judging comprises:
searching a device data storage storing information concerning a terminal whose obtained image was judged to be satisfied with said first or said second condition, and information concerning a valid period of said judgment, to confirm whether or not said judgment as to said diagnosis requesting side terminal is carried out within the corresponding valid period, and
wherein said second judging comprises:
searching said device data storage to confirm whether or said judgment as to said diagnosis side terminal is carried out within the corresponding valid period.

5. The authentication processing method as set forth in claim 1, wherein said first judging comprises:
receiving image data photographed by a camera connected to said diagnosis requesting side terminal from said diagnosis requesting side terminal;
calculating a value of a predetermined image feature amount; and
confirming whether or not said value of said predetermined image feature amount is within a prescribed range.

6. The authentication processing method as set forth in claim 1, wherein said first judging comprises:
receiving a value of a predetermined image feature amount, which calculated from image data photographed by a camera connected to said diagnosis requesting side terminal, from said diagnosis requesting side terminal;
confirming whether or not said value of said predetermined image feature amount is within a prescribed range.

7. The authentication processing method as set forth in claim 1, wherein said second judging comprises:
receiving image quality information obtained by an image sensor connected to said diagnosis side terminal from said diagnosis side terminal;
calculating a value of a predetermined image feature amount by using said image quality information; and
confirming whether or not said value of said predetermined image feature amount is within a prescribed range.

8. The authentication processing method as set forth in claim 1, wherein said second judging comprises:
receiving a value of a predetermined image feature amount calculated from image quality information obtained by an image sensor connected to said diagnosis side terminal from said diagnosis side terminal; and
confirming whether or not said value of said predetermined image feature amount is within a prescribed range.

9. The authentication processing method as set forth in claim 1, further comprising:
carrying out an environment authentication processing for said diagnosis requesting side terminal; and
carrying out an environment authentication processing for said diagnosis side terminal, and
wherein said first judging is carried out after it is detected that a processing result of said environment authentication processing for said diagnosis requesting side terminal is affirmative, and
said second judging is carried out after it is detected that a processing result of said environment authentication processing for said diagnosis side terminal is affirmative.

10. The authentication processing method as set forth in claim 5, wherein said predetermined image feature amount is a color temperature.

11. A program embodied on a medium, for causing an authentication server communicating with a diagnosis requesting side terminal and a diagnosis side terminal to execute an authentication processing, comprising:
at beginning of a remote diagnosis carried out between said diagnosis requesting side terminal and said diagnosis side terminal, judging whether or not an image obtained in said diagnosis requesting side terminal satisfies a first condition;
at the beginning of said remote diagnosis, judging whether or not an image obtained by said diagnosis side terminal satisfies a second condition; and
upon detection that affirmative judgments are obtained in the first and second judgings, generating authentication data including information concerning said diagnosis requesting side terminal, information concerning said diagnosis side terminal and a diagnosis time, and storing the generated authentication information into an authentication data storage.

12. An authentication server communicating with a diagnosis requesting side terminal and a diagnosis side terminal, comprising:
a unit that judging whether or not an image obtained in said diagnosis requesting side terminal satisfies a first condition, at beginning of a remote diagnosis carried out between said diagnosis requesting side terminal and said diagnosis side terminal;
judging whether or not an image obtained by said diagnosis side terminal satisfies a second condition, at the beginning of said remote diagnosis; and
a unit that generates authentication data including information concerning said diagnosis requesting side terminal, information concerning said diagnosis side terminal and a diagnosis time, and stores the generated authentication information into an authentication data storage, upon detection that affirmative judgments are obtained in the first and second judgings.
